(19) United States
(12) Patent Application Publication
(10) Pub. No.: US 2006/0292042 A1
(43) Pub. Date: Dec. 28, 2006

(54) Disposable testing apparatus for questionable substances

(76) Inventor: Alex Boukas, E. Setauket, NY (US)

Correspondence Address: Thomas A. O'Rourke;Bodner & O'Rourke, LLP, 425 Broadhollow Road, Mellville, NY, 11747, US

(21) Appl. No.: 11/175,900

(22) Filed: Jul. 6, 2005

Related U.S. Application Data
(60) Provisional application No. 60/585,762, filed on Jul. 6, 2004.

(51) Int. Cl. G01N 33/00

(52) U.S. Cl. 422/83; 422/68.1

(57) ABSTRACT

An apparatus for use in screening questionable substances is disclosed. The apparatus includes a means for receiving a canister of compressed gas. The canister has a discharge valve for releasing the compressed gas. The discharge valve is connected to a tube by means of an export duct, the export duct is aligned so that gas from said canister passes through the discharge valve into the export duct and then into tubing where it passes from the rear end of said tubing. The flow of gas from the export duct into the tubing creates a suction at the forward end of the tubing thereby causing a substance to be ingested into the forward end of the tubing and causing the substance to be passed from the rear end of the tubing.

This application is a conversion of United States Provisional Patent Application Ser. No. 60/585,762 filed Jul. 6, 2004, the disclosures of which are incorporated herein by reference.

### FIELD OF THE INVENTION

The present invention relates to improvements in testing devices for determining in a safe and expeditious manner whether a substance encountered in the field or elsewhere may be deleterious.

### BACKGROUND OF THE INVENTION

The analysis of unknown substances can be a dangerous and generally time consuming procedure. This analysis also typically requires specialized equipment. With the recent increase in global terrorism military actions where there is an increased risk of encountering toxic substances, for example anthrax and ricin, there is a need for cheap portable devices capable of quickly containing and testing unknown substances to ascertain the risk of danger to the public, government workers, the military, etc.

With the recent acceleration of terrorist activities around the world first responders such as police, firefighters and emergency medical personnel need a device capable of telling them what substance, if any, the person they are treating has been exposed. The need for a substance analysis device for first responders extends beyond terrorist attacks to events including but not limited to natural disasters, truck crashes, and industrial accidents. These events can disperse dangerous substances and make prompt identification nearly impossible by removing or destroying written descriptions, warning labels, etc. of said substances. Without being informed of what the substance they are dealing with is, first responders put themselves, their families and others at risk of being contaminated as well as the person they are treating. It is also necessary that a first responder know what the person they are treating has been exposed to because they cannot properly treat a person without knowing what is causing the person's condition and if first responders do not know what substance a person has been exposed to they might treat that person with a drug which will react with the unknown substance in a negative way.

First responders are not the only people who need quick and efficient analysis of unknown substances. Other examples of people who need to have unknown substances analyzed quickly include but are not limited to a soldier inspecting an enemy bunker, a waste clean up person cleaning up an industrial site, a mail room clerk who sees a package leaking a fluid, a police officer who has found an unknown substance in a suspect's car, and a teacher who has found a child unconscious next to an unmarked bottle of chemicals.

Current devices used to test the unknown substance are often times reusable and have to be decontaminated after each to prevent cross contamination and because it is uneconomical to buy a new device for every test. Decontaminating such an apparatus is labor intensive and a time consuming task which increases the cost and decrease the number of tests a reusable machine can perform. If a device is not properly decontaminated after a test than all later tests performed in the future could come up with a false positive, thus wasting time and money on cleaning up a substance which was not actually toxic.

In the cases when unknown substances are spilled in the presence of flammable or explosive compounds then most electric driven portable devices cannot be used to analyze the substance. The risk of the battery or electric motor inside of an electrically driven portable device creating a spark in an environment with flammable materials means that in that on site evaluation of the unknown substance is much more difficult.

Current substance analysis devices are large, complex machines which require a number of highly trained individuals in a laboratory to prepare samples brought in from the field for analysis, operate the machine and to interpret the data which is produced. On site analysis of unknown substances reduces the possibility of further contamination of areas surrounding the unknown substance. Current devices capable of testing substances only test a small portion of the substance leaving the rest of the substance exposed to the environment. The longer a dangerous substance is exposed to the environment the more likely it is that more people will be contaminated and that the contamination will spread.

### OBJECTS OF THE INVENTION

It is an object of the present invention to provide a portable device for the analysis of unknown substances.

It is an object of the present invention to provide a portable device which can be held in a single hand and is human powered for the analysis of unknown substances.

It is an object of the present invention to provide a portable device for the analysis of unknown substances which is portable, cost effective and easy to maintain.

It is an object of the present invention to provide a portable device for the analysis of unknown substances which will allow first responders to emergencies to operate more effectively and with greater confidence.

It is an object of the present invention to provide a portable device for the analysis of unknown substances which can operate without creating the risk of igniting flammable materials.

It is an object of the present invention to provide a portable device for the analysis of unknown substances which will give people with no special training the ability to determine if they are at risk of being contaminated by a toxic or dangerous substances.

### SUMMARY OF THE INVENTION

The present invention is directed to a device that will facilitate chemical analysis applications, specifically the identification of unknown substances. The unknown substance analysis device is primarily useful for suctioning solid, fluid or gaseous materials that the user of the present invention wishes to identify. The portability and sturdy design of the device increases its utility by allowing first responder emergency personnel and others to use a lightweight substance analysis device.

In one embodiment, the device comprises a tube attached to a body which houses one or more cans of compressed gas. The release valve of each can is connected to the tube via an export duct. A release means is attached at one end of the export duct, and at the other end to a trigger. When pressure is applied to the trigger, the release means pulls the export duct and opens the release valves on the cans. The compressed gas from the cans moves through the export duct into and out of a rear end of the tube. The flow of compressed gas causes lower pressure at the forward end of the tube. Accordingly, the forward end of the tube, a suction effect is created. The forward end of the tube allows for suitable attachments for various applications, such as elongated plastic tubes or rigid pipes. In this embodiment, the trigger is disposed on the bottom of the body, and the body is hand-held.

In another embodiment, the trigger is disposed on top of the body, and the body only houses one can of compressed air. When the trigger is depressed, the release valve feeds compressed air into the export duct. The export duct is connected to the tube, and the air flow creates a suction effect at the forward end of the tube.

In each embodiment, the forward end of the tube may be fitted with attachments, and the rear end of the tube may be fitted with attachments or a collecting pouch as desired.

In each embodiment, the cans of compressed gas may be encased in resistor wire.

The resistor wire can be attached to a battery, which will cause the resistor wire to heat the cans of compressed gas. Heating the cans of compressed gas will increase the pressure of the gas inside the cans, and thus, maintain a constant suction effect. Alternatively, a phase change material (PCM) may be used to provide heat within the can. Heat in the can will cause the gas to expand, and it will maintain a constant suction effect as the can is being evacuated.

### BRIEF DESCRIPTION OF THE DRAWINGS

FIG. 1 is a vertical cross-sectional view of a multiple canister embodiment of the present invention.

FIG. 2 is a vertical cross-sectional view of a single-canister embodiment of the present invention.

FIG. 3 is a front view of the device shown in FIG. 2.

FIG. 4 is a top view of the device shown in FIG. 2.

FIG. 5 is a rear perspective view of the device shown in FIG. 2.

FIG. 6 is a view of a single-canister embodiment of the present invention without the ring clip on the tube.

FIG. 7 is an front view of the embodiment of FIG. 6.

FIG. 8 is a vertical cross-sectional view of a single-canister embodiment of FIG. 6.

FIG. 9 is a perspective view of the embodiment of FIG. 6.

FIG. 10 is a top view of the embodiment of FIG. 6.

### DETAILED DESCRIPTION OF THE DRAWINGS

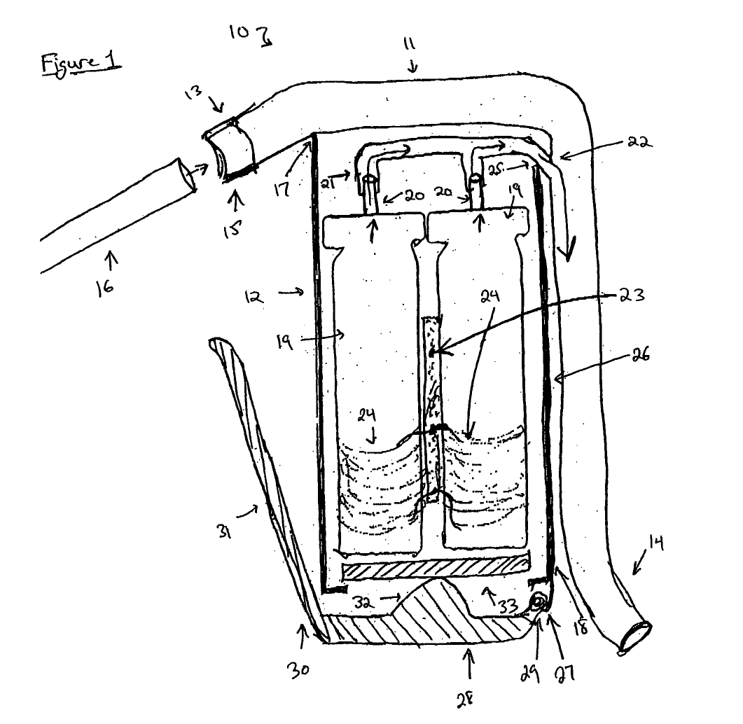

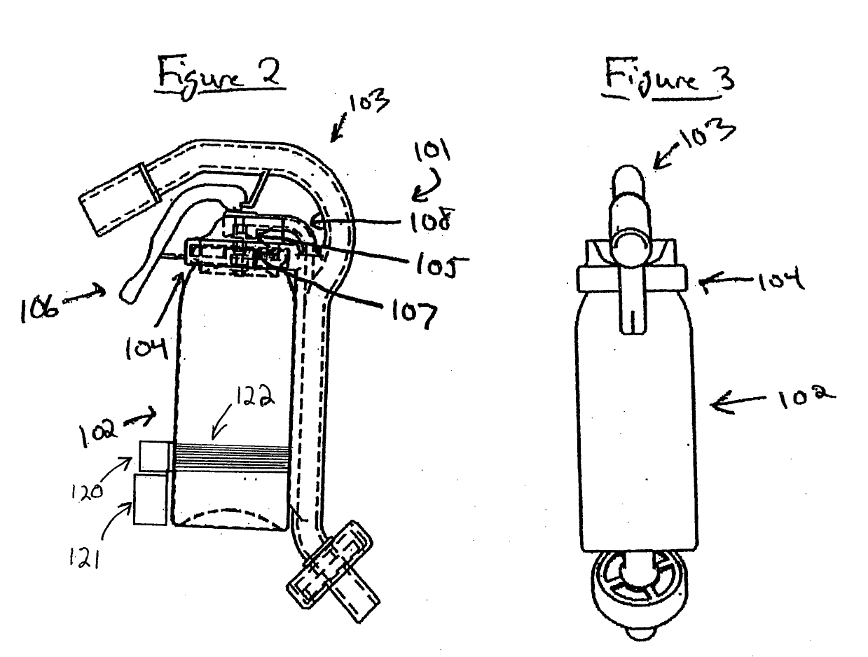

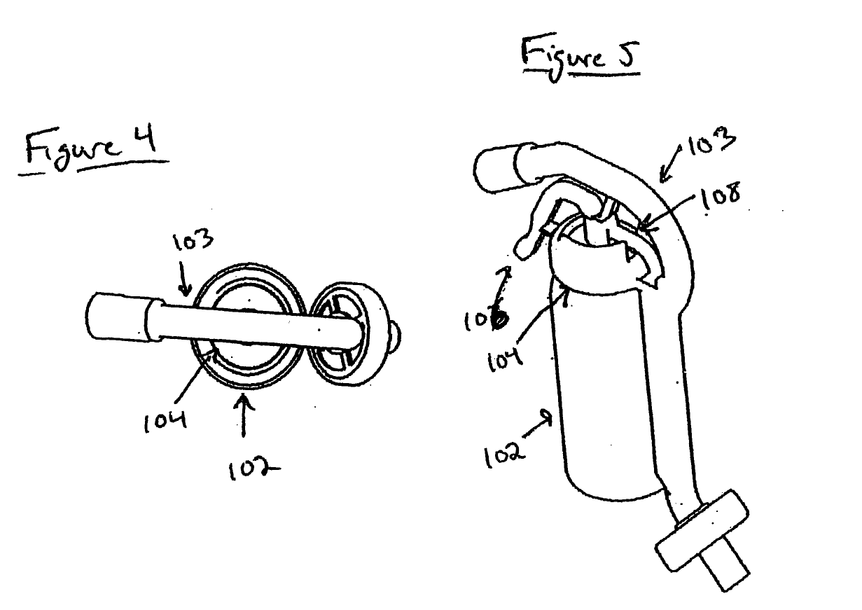

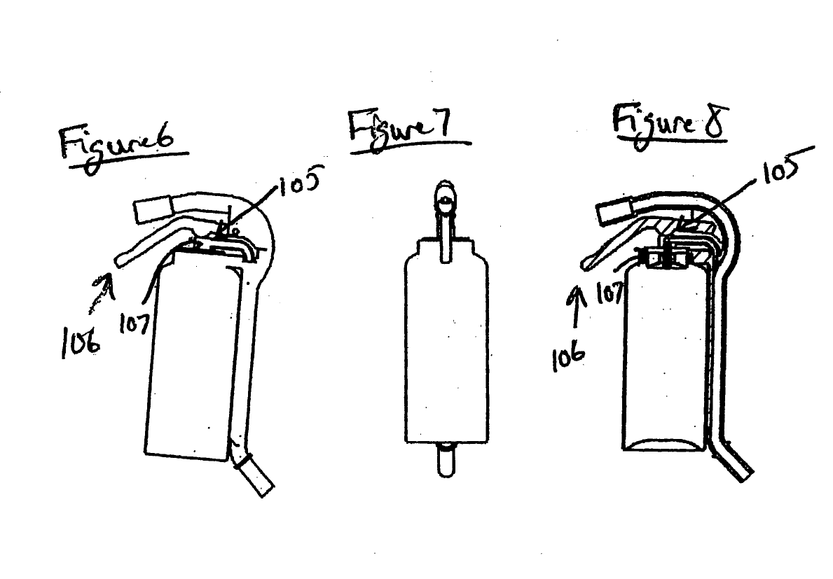

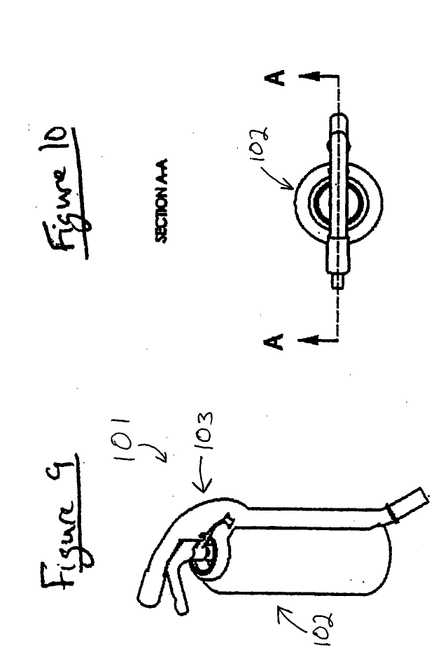

In FIG. 1, the substance collection apparatus is generally shown at 10. A tube 11 is generally hollow along its length and may be any desired length. The first section of the tube may be made of any suitable material. The tube extends along the upper surface of a body 12. The second section of the tube is generally transverse to the direction of the first section of tube and extends along the rear surface of the body 12. One preferred material is a clear, flexible plastic. The tube may be attached to a body 12 by any suitable means. Examples of suitable means can include a hook and claw type material such as Velcro®, an adhesive or adhesive tape, or other means. In addition, the tube may be part of a cap that conforms in shape to the top of the body and goes over the top of the body and which can be replaced as necessary. The tube 11 has a forward end 13 that acts as an intake and a rear end 14 for exhaust. On the forward end 13 of the tube 11 may be an attachment coupling 15, for example, for securing a length of tubing. Various attachment couplings are known in the art. In this example, the coupling can be a ring or sleeve that creates a frictional fit between the inner surface of the sleeve or ring and the outer surface of the tube. Alternatively, any suitable and adhesive material can be used. When needed, an attachment 16 may be fitted to the forward end 13 of tube 11. In one example, the attachment can have a tube portion that is set into one end of the coupling. The coupling 15 will provide a vacuum seal between the tube and the attachment.

The coupling 15 may be made of any suitable material, such as metal, plastic or rubber. Tube 11 can be fixedly or removably attached to body 12. Tube 11 may be made of any suitable material for handling gases, liquids and small solids. As seen in FIG. 1, tube 11 can be attached to the top surface 17 and/or side surface 18 of body 12. However, the tube 11 preferably need only be attached to the body 12 while the device is being used. For example, when cleaning or replacing the tube, it may be removed from the body.

The body 12 is preferably substantially hollow and houses one or more cans of compressed gas 19. In one preferred embodiment, there may be multiple canisters or cans of compressed gas. Each can of gas may be removable for ready replacement, if desired. Alternatively, the device may be a single use device that can be disposed of, if desired. Each can of gas 19 has a release valve 20, which are all connected to the tube 11 by an export duct 21. When activated, the release valves feed compressed gas into the export duct, which passes into tube 11 at inlet or port 22. The export duct may be made of any suitable tubing material for handling a gaseous material.

There is preferably a battery in the device. In one embodiment, the battery 23 can be between the cans of compressed gas 19. However, the location of the battery can vary depending on the size of the gas canisters. The battery 23 may be connected to resister wire 24 which is wrapped around at least a portion of each can. When activated, the current from the battery in the resister wire heats the cans to increase the pressure of the gas inside the cans and volume of the gas when it is released from the canisters thereby increasing the suction created by the intake. Heat sensitive fuse blocks (not shown) may be employed to prevent overheating of the canisters. Although the device is shown in a battery operated form, any source of electrical power can be used to provide the heat to the canisters. Also, it will be appreciated that the heat to the canisters could be applied by other means as well. For example, the heat can be caused by an exothermic chemical reaction in the container or heat from the sun.

In another embodiment, the battery and resistor wire may be eliminated. In this embodiment, a phase change material (PCM) may be used in the can of compressed gas. The PCM should be a material that is capable of latent heat storage. A PCM is a material that will stay at relatively the same temperature during phase change. For example, PCM's absorb and retain heat when changing from solid to liquid, but release heat when changing from liquid to solid. Some PCMs that can be used with the can of compressed gas can include, but are not limited to, salt-hydrates, paraffin waxes, normal paraffins, fatty acids and Fischer-Tropsch hard waxes. Preferably, the PCMs have a melting point between about −3 degrees Celsius and 100 degrees Celsius. When compressed gas is expelled from the can and the temperature in the can decreases due to the decrease in pressure of the gas contained in the canister, the PCM will release heat which will increase the pressure in the canister thereby keeping the suction effect constant. The phase change material may be in the canister or may be adjacent to the outer surface of the canister.

An activation means 26 runs along side surface 18 of body 12. At one end 25, the activation means 26 may be in the vicinity of or attached to export duct 21. At a second end 27, the activation means 26 may be attached to a first end 29 of a trigger 28. At a second end 30 of trigger 28 is a depression means 31. Depression means 31 may be molded as a hand-grip, with finger indentations to allow the user to grip and squeeze easily. The trigger 28 is operated as a lever with fulcrum 32 resting, or hingedly attached, to the bottom surface 33 of body 12.

In operation, the user may wrap his/her hand around the tube, body and trigger, or alternatively, around the body and trigger leaving the tube to hang over the back of the hand. As the depression means 31 is squeezed toward the body, activation means 26 pulls export duct 21 thereby opening the release valve 20 in the canister. When the export duct 21 is pulled and opened, the release valves 20 allow compressed gas to escape. The compressed air flows through the export duct 21 and out of the rear end 14 of tube 11. The rapid flow of compressed gas out of the rear end 14 of tube 11 creates a partial vacuum or suction effect at the forward end 13 of tube 11. As the depression means 31 is squeezed closer to the body 12, more compressed gas is released from the rear end 14 of tube 11, creating a greater suction effect. Thus, the user can temper the amount of suction from lightly suctioning toxic gas to removing debris particles by applying different amounts of pressure to depression means 31. The lightweightedness, hand-held operation, and manual operability make this device an optimum tool for people including but not limited to emergency personnel. The rear end of tube 14 can be secured to any suitable storage means for receiving the debris or gas sucked into the tube 11. FIG. 1 shows one means for activating the gas canisters through the use of the depression means 31. Other activating means can be used as well. For example, the activating means can be a push button, a plunger or other device for causing the release valves 20 to discharge gas.

FIG. 2 depicts another embodiment of the present invention at 101. In this embodiment, only a single can of compressed gas 102 is preferably utilized. While the tube 103 placement with the intake opening is similar to the previous embodiment, the release of compressed gas differs.

The apparatus of the present invention 101 attaches to the can of compressed gas 102 by an airtight cap 104. The attachment may be made by a snap fit, a thread connection, etc. The cap 104 encapsulates the release valve 105 on the top of the can of compressed gas. The cap can be kept air tight by an adhesive, a gasket, a sealant or other suitable means. In this embodiment, a trigger 106 is more directly attached to the release valve 105. Hence, when the trigger 106 is depressed, by a finger in this embodiment, the release valve 105 is caused to be opened into an airtight chamber or pocket 107. The pocket 107 maintains the airtight environment from the can and the pressure in the pocket forces the compressed gas out of an export duct 108 into tube 103. As the air is forced out of the tube 103 by the release of the compressed gas, a suction effect is created at the intake. The amount of suction can be controlled by the amount of pressure placed on the trigger 106. The more gas that is released from the canister into the chamber and out the end 108 of the tube, the greater the suction effect. The compressed gas flows from the export duct 108 into the tubing 103. As the compressed gas passes from the rear end of the tubing, any material that enters the front end of the tubing will flow through the tubing and can be collected by a suitable receptacle.

In this embodiment, a battery 121 may be connected to a resister wire 122, which may be wrapped around the can of compressed gas 102. When activated, the current in the resister wire 122 heats the cans to increase the pressure of the gas inside the cans and thus increase the suction created as the gas is discharged. One or more heat sensitive fuse blocks 120 may also be employed to prevent overheating as discussed above. Additionally, the resistor wire and can 102 may be covered by an insulating material (not shown). The insulating material will effectively contain the heat created by the activation of the resistor wire 122. Also, the insulating material will protect the user's hand from any uncomfortable temperature changes that may be associated with activation of the battery 121. Also, in this embodiment, a PCM may be used in substantially the same manner as described above.

It is understood that the body, tube and can of compressed gas may be of any size, shape or material. Preferably, the tube will be a soft plastic and body will be hard plastic for easy maintenance and cleaning. Additionally, the body may have a clip for attachment to a belt or strap of a bag. Also, the cans of compressed gas will be easily replaceable in the present invention, by either removing them from the body, or removing the cap.

In any embodiment, a container or other receptacle may be attached to the rear end 14 of the tube 11. Preferably, the container is a bag or pouch that will enable the user to collect any fluid that is suctioned through tube 11. The container may have an open end, which will preferably create an airtight seal around the rear end 14 of tube 11. In some manner, the bag may allow gas to escape preferably through a filter, while suctioning liquid, debris or polluted air. In one embodiment, the container will be equipped with an attachment port, which can mate with a filter. The filter may allow gas to escape, while retaining liquid, debris or polluted gas. Wher a filter is used the air (i.e. gas) filtration device employed should have a pore diameter sufficient to entrap the chosen micro-organism. For example, in the case of anthrax (Bacillus anthracis) which has a diameter of 1 urn, a pore size of e.g. 0.2 pm may be used. The air filtration device may also comprise a fan to encourage the flow of air through the filter, and a large volume of air can be passed through the filter in a relatively small period of time, all particulate matter of a greater size than the pore diameter being retained. The filter may then be contacted with a sample fluid (e.g. a buffer or another solution, fluid or mixture into which the given micro-organism can be suspended or solubilised) if the testing so requires.

In another embodiment, the container itself may be manufactured from a gas-permeable, liquid-retaining compound. In a further embodiment, the container may be equipped with vents that will allow gas to escape. In each embodiment, the container may be equipped with a sealing means, which will allow the user to remove and seal the container. A sealing means, which may be an adhesive strip or cap, can be used if the container houses hazardous material, or a liquid that can be reused after being filtered.

The present invention is particularly suitable for environmental testing, i.e., the environment (where exposure may have occurred). During the course of an investigation important information may be obtained from sampling the environment where the exposure may have occurred. Testing of the environment is useful for detecting trace amounts of anthrax spores. Specimens obtained may include:

Samples of the air or other gases.

Material on various surfaces that are suctioned into the device.

These samples are then later processed in the laboratory for testing or to promote growth of any spores, if present to ascertain the identity of the material obtained. If suspicious bacteria grow, additional testing—like that conducted on suspect powders—also may be performed.

Lab testing of powders or other materials suspected of harboring the anthrax bacteria is often an important component of an investigation. Tests may include:

Cultures of suspected materials.

Microscopic examination of a sample of a suspect material.

Valuation of the characteristics of the suspect agent's growth properties.

PCR (polymerase chain reaction) test that amplifies trace amounts of DNA to document that the bacteria is present.

DFA (direct fluorescent assay) to detect key bacterial proteins.

Other specialized tests to confirm the identification of the bacteria.

The specimens are picked up by the device of the present invention and can flow through the tubing and be exhausted out the end of the tubing into a receptacle (not shown) which can be sealed and removed from the tubing and sent for testing.

I claim:

1. An apparatus for use in screening questionable substances comprising a canister of compressed gas, said canister having a discharge valve for releasing said compressed gas, said discharge valve being connected to a tube by means of an export duct, said tube having a forward end and a rear end, said export duct being aligned so that gas from said canister passes through said discharge valve into said export duct and then into said tubing where it passes from said rear end of said tubing and wherein the flow of gas from said export duct into said tubing creates a suction at the forward end of said tubing thereby causing a substance to be ingested into the forward end of the tubing and causing said substance to be passed from said rear end of said tubing.

2. The apparatus according to claim 1 wherein said substance is a gas.

3. The apparatus according to claim 1 wherein said substance is a powder.

4. The apparatus according to claim 1 wherein there are a plurality of canisters.

5. The apparatus according to claim 1 further comprising a means for heating the compressed gas in said canister to keep the suction constant.

6. The apparatus according to claim 5 wherein said means is a heating coil

7. The apparatus according to claim 5 wherein said means is a phase change material.

8. The apparatus according to claim 5 wherein there is an activation means for initiating a release of gas from the canister.

9. The apparatus according to claim 8 wherein said activation means is pressure operated.

10. The apparatus according to claim 9 wherein said activation means has a first end attached to the export duct and a second end attached to a trigger.

11. The apparatus according to claim 9 wherein said activation means has a first end attached to a said release valve.

12. The apparatus according to claim 10 wherein said trigger has a depression means and a fulcrum for manipulating the activation means.

13. The apparatus according to claim 1 wherein a cap encapsulates the release valve and a trigger causes said release valve to discharge gas into a chamber in said cap, said gas passing from said chamber to said export duct where it passes to said tube thereby causing a suction at said forward end of said tube.

14. An apparatus for use in screening questionable substances comprising a a means for receiving a canister of compressed gas, said canister having a discharge valve for releasing said compressed gas, said discharge valve being connected to a tube by means of an export duct, said tube having a forward end and a rear end, said export duct being aligned so that gas from said canister passes through said discharge valve into said export duct and then into said tubing where it passes from said rear end of said tubing and wherein the flow of gas from said export duct into said tubing creates a suction at the forward end of said tubing thereby causing a substance to be ingested into the forward end of the tubing and causing said substance to be passed from said rear end of said tubing.
